(19) United States
(12) Patent Application Publication
(10) Pub. No.: US 2024/0010632 A1
(43) Pub. Date: Jan. 11, 2024

(54) SOLID STATE FORMS OF ERDAFITINIB SALTS AND PROCESSES FOR PREPARATION OF ERDAFITINIB

(71) Applicant: TEVA PHARMACEUTICALS INTERNATIONAL GMBH, Jona (CH)

(72) Inventors: Zuzana Przeczkova, Ostrava (CZ); Adéla Bártová, Ostrava-Michalkovice (CZ); Pavel Kolesa, Hlubocec (CZ); Ondrej Skarka, Brno (CZ); Jirí Faustmann, Opava-6 (CZ); Zdenek Horák, Opava (CZ); Zdenek Jelínek, Ostrov U Macochy (CZ); Pavlína Horáková, Bochor (CZ)

(21) Appl. No.: 18/021,238

(22) PCT Filed: Aug. 17, 2021
(86) PCT No.: PCT/US2021/046194

§ 371 Date: Feb. 14, 2023

Related U.S. Application Data
(60) Provisional application No. 63/090,822, filed on Oct. 13, 2020.
(60) Provisional application No. 63/066,385, filed on Aug. 17, 2020.

(51) Int. Cl. C07D 403/04; C07F 7/18

(52) U.S. Cl. CPC C07D 403/04; C07F 7/1804; C07B 2200/13

(57) ABSTRACT

The present disclosure relates to solid state forms of Erdafitinib salts, processes for preparation thereof, processes for preparation of Erdafitinib and pharmaceutical compositions thereof.

### TECHNICAL FIELD

The present disclosure relates to solid state forms of Erdafitinib salts, processes for preparation thereof, processes for preparation of Erdafitinib and pharmaceutical compositions thereof.

### BACKGROUND

Erdafitinib has the chemical name N-(3,5-dimethoxyphenyl)-N′-(1-methylethyl)-N-[3-(1-methyl-1H-pyrazol-4-yl-)quinoxalin-6-yl]ethane-1,2-diamine. Erdafitinib has the following chemical structure:

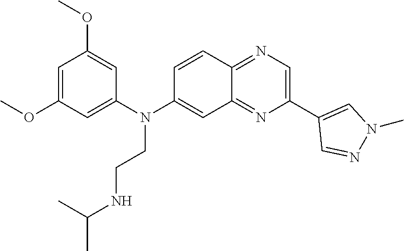

Erdafitinib is a fibroblast growth factor receptor (FGFR) inhibitor with potential antineoplastic activity that may be used for the treatment of patients with locally advanced or metastatic urothelial cancer (UC) and certain fibroblast growth factor receptor (FGFR) genetic alterations whose tumors have progressed after prior chemotherapy. Erdafitinib is also under investigation for treatment of advanced solid tumors with fibroblast growth factor receptor (FGFR) mutations and gene fusions, and refractory solid tumors including cholangiocarcinoma.

Erdafitinib is disclosed in International Publication No. WO 2011/135376.

Polymorphism, the occurrence of different crystal forms, is a property of some molecules and molecular complexes. A single compound, like Erdafitinib, may give rise to a variety of polymorphs having distinct crystal structures and physical properties like melting point, thermal behaviors (e.g., measured by thermogravimetric analysis—“TGA”, or differential scanning calorimetry—“DSC”), X-ray powder diffraction (XRPD) pattern, infrared absorption fingerprint, Raman absorption fingerprint, and solid state (13C-) NMR spectrum. One or more of these techniques may be used to distinguish different polymorphic forms of a compound.

Different solid state forms (including solvated forms) of an active pharmaceutical ingredient may possess different properties. Such variations in the properties of different solid state forms and solvates may provide a basis for improving formulation, for example, by facilitating better processing or handling characteristics, improving the dissolution profile, or improving stability (polymorph as well as chemical stability) and shelf-life. These variations in the properties of different solid state forms may also provide improvements to the final dosage form, for instance, if they serve to improve bioavailability. Different solid state forms and solvates of an active pharmaceutical ingredient may also give rise to a variety of polymorphs or crystalline forms, which may in turn provide additional opportunities to use variations in the properties and characteristics of a solid active pharmaceutical ingredient for providing an improved product.

Discovering new solid state forms and solvates of a pharmaceutical product can provide materials having desirable processing properties, such as ease of handling, ease of processing, storage stability, and ease of purification or as desirable intermediate crystal forms that facilitate conversion to other polymorphic forms. New polymorphic forms and solvates of a pharmaceutically useful compound can also provide an opportunity to improve the performance characteristics of a pharmaceutical product (dissolution profile, bioavailability, etc.). It enlarges the repertoire of materials that a formulation scientist has available for formulation optimization, for example by providing a product with different properties, e.g., a different crystal habit, higher crystallinity or polymorphic stability which may offer better processing or handling characteristics, improved dissolution profile, or improved shelf-life.

For at least these reasons, there is a need for additional salts and solid state forms (including solvated forms) of Erdafitinib.

### SUMMARY

The present disclosure relates Erdafitinib salts such as Erdafitinib acetate, Erdafitinib formate and Erdafitinib mesylate and solid state forms thereof. The present disclosure also relates to processes for preparation of the salts and solid state forms thereof, and pharmaceutical compositions including the solid state forms.

The present disclosure also provides uses of the said solid state forms of Erdafitinib salts such as Erdafitinib acetate, Erdafitinib formate and Erdafitinib mesylate in the preparation of other solid state forms of Erdafitinib or other salts thereof.

The present disclosure also provides the said solid state forms of Erdafitinib salts such as Erdafitinib acetate, Erdafitinib formate and Erdafitinib mesylate for use in the preparation of other solid state forms of Erdafitinib or other salts thereof.

In another embodiment, the present disclosure encompasses use of the described solid state forms of Erdafitinib salts such as Erdafitinib acetate, Erdafitinib formate and Erdafitinib mesylate in the preparation of pharmaceutical compositions and/or formulations, optionally for the treatment of patients with locally advanced or metastatic urothelial cancer (UC) and certain fibroblast growth factor receptor (FGFR) genetic alterations whose tumors have progressed after prior chemotherapy, advanced solid tumors with fibroblast growth factor receptor (FGFR) mutations and gene fusions, and refractory solid tumors including cholangiocarcinoma.

In another embodiment, the present disclosure encompasses the described solid state forms of Erdafitinib salts such as Erdafitinib acetate, Erdafitinib formate and Erdafitinib mesylate for use in the preparation of pharmaceutical compositions and/or formulations, optionally for the treatment of patients with locally advanced or metastatic urothelial cancer (UC) and certain fibroblast growth factor receptor (FGFR) genetic alterations whose tumors have progressed after prior chemotherapy, advanced solid tumors with fibroblast growth factor receptor (FGFR) mutations and gene fusions, and refractory solid tumors including cholangiocarcinoma.

The present disclosure further provides pharmaceutical compositions including the solid state forms of Erdafitinib salts such as Erdafitinib acetate, Erdafitinib formate and Erdafitinib mesylate according to the present disclosure.

In yet another embodiment, the present disclosure encompasses pharmaceutical formulations including the described solid state forms of Erdafitinib salts such as Erdafitinib acetate, Erdafitinib formate and Erdafitinib mesylate and at least one pharmaceutically acceptable excipient.

The present disclosure encompasses processes to prepare said pharmaceutical formulations of Erdafitinib including combining the described solid state forms of Erdafitinib salts such as Erdafitinib acetate, Erdafitinib formate and Erdafitinib mesylate and at least one pharmaceutically acceptable excipient.

The solid state forms defined herein as well as the pharmaceutical compositions or formulations of the improved solid state form of Erdafitinib salts such as Erdafitinib acetate, Erdafitinib formate and Erdafitinib mesylate can be used as medicaments, in embodiments for the treatment of patients with locally advanced or metastatic urothelial cancer (UC) and certain fibroblast growth factor receptor (FGFR) genetic alterations whose tumors have progressed after prior chemotherapy, advanced solid tumors with fibroblast growth factor receptor (FGFR) mutations and gene fusions, and refractory solid tumors including cholangiocarcinoma.

The present disclosure also provides methods of treating medicaments, in embodiments for the treatment of cancer as specified above, by administering a therapeutically effective amount of the improved solid state form of Erdafitinib salts such as Erdafitinib acetate, Erdafitinib formate and Erdafitinib mesylate of the present disclosure, or at least one of the herein described pharmaceutical compositions or formulations, to a subject suffering from the above specified diseases, or otherwise in need of the treatment.

The present disclosure also provides uses of the improved solid state form of Erdafitinib salts such as Erdafitinib acetate, Erdafitinib formate and Erdafitinib mesylate of the present disclosure, or at least one of the above pharmaceutical compositions or formulations for the manufacture of medicaments, in embodiments for medicaments, in embodiments for the treatment of patients with locally advanced or metastatic urothelial cancer (UC) and certain fibroblast growth factor receptor (FGFR) genetic alterations whose tumors have progressed after prior chemotherapy, advanced solid tumors with fibroblast growth factor receptor (FGFR) mutations and gene fusions, and refractory solid tumors including cholangiocarcinoma.

### BRIEF DESCRIPTION OF THE FIGURES

FIG. 1 shows an X-ray powder diffractogram (XRPD) of crystalline Form P of Erdafitinib acetate.

FIG. 2 shows an X-ray powder diffractogram (XRPD) of crystalline Form B of Erdafitinib formate.

FIG. 3 shows an X-ray powder diffractogram (XRPD) of crystalline Form D of Erdafitinib formate.

FIG. 4 shows an X-ray powder diffractogram (XRPD) of crystalline Form F of Erdafitinib mesylate.

### DETAILED DESCRIPTION

The present disclosure relates to improved solid state forms of Erdafitinib salts such as Erdafitinib acetate, Erdafitinib formate and Erdafitinib mesylate. The present disclosure also relates to processes for preparation thereof, and pharmaceutical compositions comprising the disclosed solid state forms.

The solid state forms of Erdafitinib salts such as Erdafitinib acetate, Erdafitinib formate and Erdafitinib mesylate according to the present disclosure may have advantageous properties selected from at least one of: chemical or polymorphic purity, flowability, solubility, dissolution rate, bioavailability, morphology or crystal habit, stability—such as chemical stability as well as thermal and mechanical stability with respect to polymorphic conversion, stability towards dehydration and/or storage stability, a lower degree of hygroscopicity, low content of residual solvents and advantageous processing and handling characteristics such as compressibility, or bulk density.

A crystal form may be referred to herein as being characterized by graphical data “as depicted in” a Figure. Such data include, for example, powder X-ray diffractograms and solid state NMR spectra. As is well-known in the art, the graphical data potentially provides additional technical information to further define the respective solid state form (a so-called “fingerprint”) which can not necessarily be described by reference to numerical values or peak positions alone. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. For example, a crystal form of Erdafitinib acetate referred to herein as being characterized by graphical data “as depicted in” a Figure will thus be understood to include any crystal forms of the Erdafitinib acetate, characterized with the graphical data having such small variations, as are well known to the skilled person, in comparison with the Figure.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% or less, about 10% or less, about 5% or less, about 2% or less, about 1% or less, or 0% of any other forms of the subject compound as measured, for example, by XRPD. Thus, for example, the solid state form of Erdafitinib acetate described herein as substantially free of any other solid state forms would be understood to contain greater than about 80% (w/w), greater than about 90% (w/w), greater than about 95% (w/w), greater than about 98% (w/w), greater than about 99% (w/w), or 100% of the subject solid state form of Erdafitinib acetate. Accordingly, in some embodiments of the disclosure, the described solid state form of Erdafitinib acetate may contain from about 1% to about 20% (w/w), from about 5% to about 20% (w/w), or from about 5% to about 10% (w/w) of one or more other solid state forms of the Erdafitinib.

As used herein, unless stated otherwise, XRPD peaks reported herein are optionally measured using CuK α radiation, λ=1.54187 Å, at a temperature of 25±3° C.

As used herein, and unless stated otherwise, the term “anhydrous” in relation to crystalline Erdafitinib relates to crystalline Erdafitinib which does not include any crystalline water (or other solvents) in a defined, stoichiometric amount within the crystal. Moreover, an “anhydrous” form does not contain more than about 1% (w/w) of either water or organic solvents as measured for example by TGA.

The term “solvate”, as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to solid state form of Erdafitinib of the present disclosure corresponds to solid state form of Erdafitinib that is physically separated from the reaction mixture in which it is formed.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., about 22° C. to about 27° C., or about 25° C.

A process or step may be referred to herein as being carried out “overnight.” This refers to a time interval, e.g., for the process or step, that spans the time during the night, when that process or step may not be actively observed. This time interval is from about 8 to about 20 hours, about 10 to about 18 hours, or about 16 hours.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding methyl tert-butyl ether (MTBE) (1.5 v/v) to a 100 ml reaction mixture would indicate that 150 mL of MTBE was added.

As used herein, the term “reduced pressure” refers to a pressure of from about 10 mbar to 50 mbar.

As used herein and unless indicated otherwise, the term “ambient conditions” refer to atmospheric pressure and a temperature of 22-24° C.

The processes of the present invention advantageously provide a convenient synthesis of Erdafitinib salts, i.e., Erdafitinib acetate, Erdafitinib formate or Erdafitinib mesylate from compound 9, wherein the intermediate Erdafitinib need not be isolated as a solid (e.g., the intermediate Erdafitinib can be obtained from the amination of compound 9, without isolation as a solid, and/or without purification (such as by crystallization or chromatography) after the reaction work up. Thus, in any embodiment of this process or in any embodiment or aspect involving these process steps, the conversion of compound 9 to Erdafitinib acetate, Erdafitinib formate or Erdafitinib mesylate can be carried out without purification of Erdafitinib.

The processes of the present invention further advantageously provides a convenient synthesis of the compound 9 from compound 4, wherein one or both of the intermediate compound 8 and/or compound 15 (preferably both), need not be isolated as a solid e.g., the intermediate compound 8 can be obtained from the alkylation of compound 9, and/or the subsequent deprotection of intermediate compound 15 can be obtained from compound 8, without isolation of either one or both of compounds 8 and 15 as solids, and/or without purification of compounds 8 and 15 (such as by crystallization or chromatography) after the reaction work up. Thus, in any embodiment of this process or in any embodiment or aspect involving these process steps, the conversion of compound 4 to compound 8 and/or conversion of compound 8 to compound 15 to Erdafitinib formate or Erdafitinib mesylate can be carried out without purification of the intermediate compounds 8 and/or compound 15, particularly without isolation of intermediate compounds 8 and/or compound 15 as a solid. Preferably, in any embodiment of this process or in any embodiment or aspect involving these process steps, the conversion of compound 4 to compound 8 and subsequent conversion of compound 8 to compound 15 to Erdafitinib acetate, Erdafitinib formate or Erdafitinib mesylate is carried out without purification of the intermediate compounds 8 and compound 15, and more preferably without isolation of intermediates compound and compound 15 as solids.

In any embodiment of the present process, the disclosure relates to the above process wherein any one, two or all, of compounds 8, 15 and Erdafitinib (obtained by amination of compound 9) are prepared and used in the next step without further purification. Preferably, compounds 8, 15 and Erdafitinib (from compound 9) are maintained in solution during work-up and directly used as a solution in the subsequent step. Thus, in any embodiment of this process, any one, two or all, preferably all, of the compounds 8, 15 and Erdafitinib (obtained by amination of compound 9), are used in a subsequent reaction step without purification, i.e. the compound(s) are obtained as a solution from any reaction work up, or optionally obtained as a solid or wet solid after work up and solvent removal, and more preferably as a solution from reaction work-up, and used directly in a subsequent reaction step.

Unless otherwise indicated, the reference to the term “without isolation” in relation to a compound according to any aspect or embodiment of the invention, means that the compound obtained from a reaction is not subjected to purification steps, such as chromatography, or crystallization/recrystallization. Thus the term “without purification” refers to a compound that is obtained from the reaction mixture after any work-up step, but without purification. Typically, such a compound may be referred to as a “crude compound”. Such a compound may be in the form of a solid (e.g., obtained after work-up and removal of any solvent from the work up) or the compound may be in a solution obtained after work-up without removal of solvent, or as a wet solid obtained after work-up and partial removal of solvent. Preferably, the compound is maintained as a solution during work-up and used as starting material as a solution and therefore without isolation as a solid or any chromatographic or crystallization procedures.

The present disclosure includes Erdafitinib formate, Erdafitinib acetate and Erdafitinib mesylate, as well as crystalline forms thereof, pharmaceutical compositions thereof, the use of these for the preparation of pharmaceutical compositions or formulations. These salts of Erdafitinib may be used in the treatment of locally advanced or metastatic urothelial cancer (UC), fibroblast growth factor receptor (FGFR) genetic alterations wherein the tumors have progressed after prior chemotherapy, advanced solid tumors with fibroblast growth factor receptor (FGFR) mutations and gene fusions, and refractory solid tumors including cholangiocarcinoma. Alternatively, the Erdafitinib formate, Erdafitinib acetate and Erdafitinib mesylate of the present disclosure may be used as intermediates in the preparation of Erdafitinib or a solid state form thereof, or another salt of Erdafitinib or a solid state form thereof. Advantageously, the Erdafitinib formate, Erdafitinib acetate and Erdafitinib mesylate of the present disclosure may be used as intermediates for the purification of Erdafitinib or another salt of Erdafitinib.

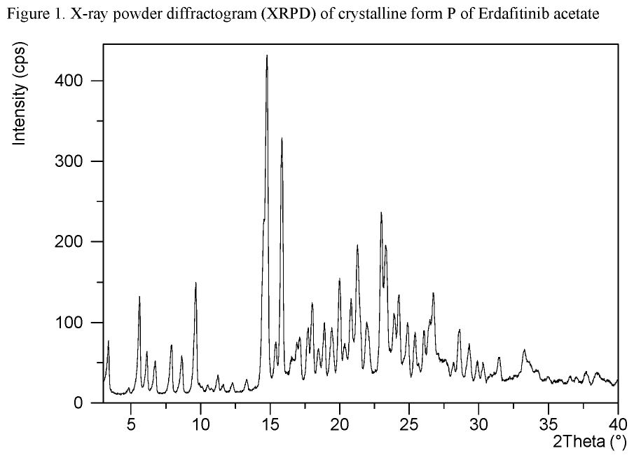

The present disclosure relates to a crystalline form of Erdafitinib acetate designated Form P. The crystalline Form P of Erdafitinib acetate may be characterized by data selected from one or more of the following: an XRPD pattern having peaks at 3.4, 5.6, 6.2, 8.7 and 9.7 degrees 2-theta±0.2 degrees 2-theta; or an XRPD pattern as depicted in FIG. 1.

Crystalline Form P of Erdafitinib acetate may be further characterized by an XRPD pattern having peaks at 3.4, 5.6, 6.2, 8.7 and 9.7 degrees 2-theta±0.2 degrees 2-theta, and also having one, two, three, four or five additional peaks selected from 6.8, 14.8, 20.0, 21.3 and 23.4 degrees two theta±0.2 degrees two theta.

According to any aspect or embodiment of the present disclosure, crystalline Form P of Erdafitinib acetate may be alternatively characterized by an XRPD pattern having peaks at 3.4, 5.6, 6.2, 6.8, 8.7, 9.7, 14.8, 20.0, 21.3 and 23.4 degrees 2-theta±0.2 degrees 2-theta.

Crystalline Form P of Erdafitinib acetate can be characterized by any combination of the above data.

In some embodiments crystalline Form P of Erdafitinib acetate may be isolated.

In some embodiments crystalline Form P of Erdafitinib acetate may be polymorphically pure.

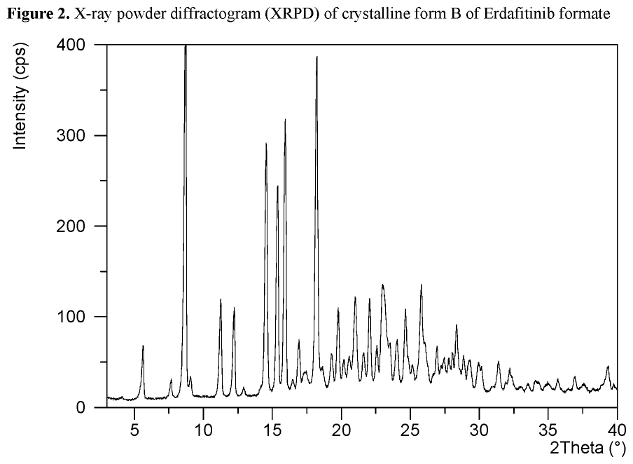

The present disclosure relates to a crystalline form of Erdafitinib formate designated Form B. The crystalline Form B of Erdafitinib formate may be characterized by data selected from one or more of the following: an XRPD pattern having peaks at 5.6, 8.7, 11.2, 14.5 and 15.4 degrees 2-theta±0.2 degrees 2-theta; or an XRPD pattern as depicted in FIG. 2.

Crystalline Form B of Erdafitinib formate may be further characterized by an XRPD pattern having peaks at 5.6, 8.7, 11.2, 14.5 and 15.4 degrees 2-theta±0.2 degrees 2-theta, and also having one, two, three, four or five additional peaks selected from 12.2, 18.2, 19.7, 22.0 and 24.6 degrees two theta±0.2 degrees two theta.

According to any aspect or embodiment of the present disclosure, crystalline Form B of Erdafitinib formate may be alternatively characterized by an XRPD pattern having peaks at 5.6, 8.7, 11.2, 12.2, 14.5, 15.4, 18.2, 19.7, 22.0, and 24.6 degrees 2-theta±0.2 degrees 2-theta.

Crystalline Form B of Erdafitinib formate can be characterized by any combination of the above data.

In some embodiments crystalline Form B of Erdafitinib formate may be isolated.

In some embodiments crystalline Form B of Erdafitinib formate may be polymorphically pure.

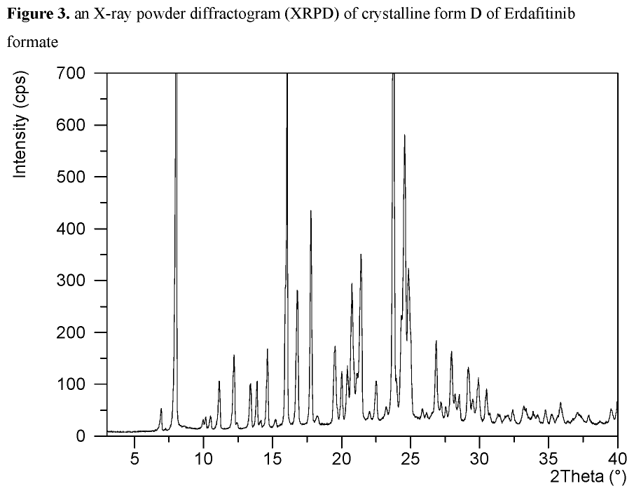

The present disclosure relates to a crystalline form of Erdafitinib formate designated Form D. The crystalline Form D of Erdafitinib formate may be characterized by data selected from one or more of the following: an XRPD pattern having peaks at 6.9, 8.0, 13.4, 17.8 and 20.7 degrees 2-theta±0.2 degrees 2-theta; or an XRPD pattern as depicted in FIG. 3.

Crystalline Form D of Erdafitinib formate may be further characterized by an XRPD pattern having peaks at 6.9, 8.0, 13.4, 17.8 and 20.7 degrees 2-theta±0.2 degrees 2-theta, and also having one, two, three, four or five additional peaks selected from 16.8, 21.4, 22.5, 23.8 and 24.6 degrees two theta±0.2 degrees two theta.

According to any aspect or embodiment of the present disclosure, crystalline Form D of Erdafitinib formate may be alternatively characterized by an XRPD pattern having peaks at 6.9, 8.0, 13.4, 16.8, 17.8, 20.7, 21.4, 22.5, 23.8, and 24.6 degrees 2-theta±0.2 degrees 2-theta.

Crystalline Form D of Erdafitinib formate can be characterized by any combination of the above data.

In some embodiments crystalline Form D of Erdafitinib formate may be isolated.

In some embodiments crystalline Form D of Erdafitinib formate may be polymorphically pure.

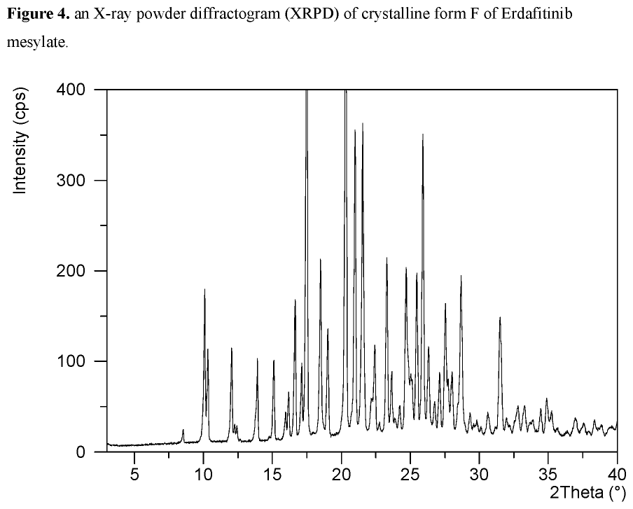

The present disclosure relates to a crystalline form of Erdafitinib mesylate designated Form F. The crystalline Form F of Erdafitinib mesylate may be characterized by data selected from one or more of the following: an XRPD pattern having peaks at 12.0, 16.6, 17.5, 18.5 and 20.3 degrees 2-theta±0.2 degrees 2-theta; or an XRPD pattern as depicted in FIG. 4.

Crystalline Form F of Erdafitinib mesylate may be further characterized by an XRPD pattern having peaks at 12.0, 16.6, 17.5, 18.5 and 20.3 degrees 2-theta±0.2 degrees 2-theta, and also having one, two, three, four or five additional peaks selected from 17.1, 19.0, 21.0, 21.5 and 23.3 degrees two theta±0.2 degrees two theta.

According to any aspect or embodiment of the present disclosure, crystalline Form F of Erdafitinib mesylate may be further characterized by an XRPD pattern having peaks at 12.0, 16.6, 17.1, 17.5, 18.5, 19.0, 20.3, 21.0, 21.5, and 23.3 degrees 2-theta±0.2 degrees 2-theta, and also having one, two, three, four or five additional peaks selected from, degrees two theta±0.2 degrees two theta.

Crystalline Form F of Erdafitinib mesylate can be characterized by any combination of the above data.

In some embodiments crystalline Form F of Erdafitinib mesylate may be isolated.

In some embodiments crystalline Form F of Erdafitinib mesylate may be polymorphically pure.

In any aspect or embodiment of the present disclosure, any of the solid state forms of the Erdafitinib salts described herein may be polymorphically pure or may be substantially free of any other solid state forms of the subject Erdafitinib salt, for example, form D of Erdafitinib formate may be substantially free of other solid state forms of Erdafitinib formate. In any aspect or embodiment of the present disclosure, any of the solid state forms of Erdafitinib salts described in any aspect or embodiment disclosed herein, may contain: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of any other solid state forms of the subject compound, preferably as measured by XRPD. Thus, any of the disclosed crystalline forms of Erdafitinib salts described herein may be substantially free of any other solid state forms of the subject Erdafitinib salt, and may contain greater than about 80% (w/w), greater than about 90% (w/w), greater than about 95% (w/w), greater than about 98% (w/w), greater than about 99% (w/w), or about 100% of the subject solid state form of the Erdafitinib salt.

It has been surprisingly found that Erdafitinib formate, Erdafitinib acetate and Erdafitinib mesylate, and particularly crystalline Erdafitinib acetate, preferably crystalline form P, crystalline Erdafitinib formate preferably crystalline forms B and/or D and crystalline Erdafitinib mesylate, preferably crystalline form F offer significant impurity purging capability.

The present disclosure also provides the use of the solid state form of Erdafitinib salts disclosed herein in the preparation of other solid state forms of Erdafitinib or other salts thereof.

The present disclosure also provides the said solid state forms of Erdafitinib salts for use in the preparation of other solid state forms of Erdafitinib or other salts thereof.

In another embodiment, the present disclosure encompasses use of the described solid state forms of Erdafitinib salts in the preparation of pharmaceutical compositions and/or formulations, optionally for the treatment of patients with locally advanced or metastatic urothelial cancer (UC) and certain fibroblast growth factor receptor (FGFR) genetic alterations whose tumors have progressed after prior chemotherapy, advanced solid tumors with fibroblast growth factor receptor (FGFR) mutations and gene fusions, and refractory solid tumors including cholangiocarcinoma.

In another embodiment, the present disclosure encompasses the described solid state forms of Erdafitinib salts for use in the preparation of pharmaceutical compositions and/or formulations, optionally for the treatment of patients with locally advanced or metastatic urothelial cancer (UC) and certain fibroblast growth factor receptor (FGFR) genetic alterations whose tumors have progressed after prior chemotherapy, advanced solid tumors with fibroblast growth factor receptor (FGFR) mutations and gene fusions, and refractory solid tumors including cholangiocarcinoma.

The present disclosure further provides pharmaceutical compositions including the solid state forms of Erdafitinib salts according to the present disclosure.

In yet another embodiment, the present disclosure encompasses pharmaceutical formulations including the described solid state forms of Erdafitinib salts and at least one pharmaceutically acceptable excipient.

In a further aspect, the present invention provides a process for the purification of Erdafitinib or salt of Erdafitinib, comprising:
a) preparing an acid addition salt of Erdafitinib, selected from the group consisting of Erdafitinib acetate, Erdafitinib formate, or Erdafitinib mesylate; and
b) converting the acid addition salt of Erdafitinib to Erdafitinib or another salt thereof.

Particularly, the present invention provides a process for the preparation of Erdafitinib or a salt thereof, preferably substantially pure Erdafitinib or a salt thereof, wherein the process comprises:
a) contacting Erdafitinib with acetic acid, formic acid or methane sulfonic acid to produce the acid addition salt Erdafitinib; and
b) converting the acid addition salt of Erdafitinib to Erdafitinib or another salt thereof, preferably to Erdafitinib.

The present disclosure further provides a process for preparation of Erdafitinib or salt thereof, preferably substantially pure Erdafitinib or pure salt thereof, wherein the process comprises:
a) contacting Erdafitinib with acetic acid to produce acetate salt of Erdafitinib; and
b) converting the acetate salt of Erdafitinib to Erdafitinib or another salt thereof.

In embodiments, Erdafitinib acetate formed in step a) is crystalline, preferably Erdafitinib acetate form P.

Formation of the acetate salt advantageously aids in removal of impurities prior to isolation of the freebase. The acetate salt is subsequently converted back to the freebase form (step (i)) to produce Erdafitinib.

In particular, in step (b) the acetate salt may be contacted with a base, such as an organic or inorganic base, to produce the corresponding freebase. The base may be any suitable alkaline, preferably selected from the group consisting of: sodium bicarbonate, sodium carbonate, sodium hydroxide, potassium carbonate, potassium bicarbonate, potassium hydroxide, cesium bicarbonate, cesium carbonate, cesium hydroxide, lithium bicarbonate, lithium carbonate, lithium hydroxide, ammonia, an organic amine, preferably a mono(C₁-C₆)alkylamine, a di(C₁-C₆)alkylamine, a tri(C₁-C₆)alkylamine), a tertiary(C₁-C₆)alkylamine, a C₅-C₁₀ heterocyclic amine, a C₅-C₁₀ arylamine, a C₄-C₈ heteroaryl amine, or combinations thereof. More particularly, the base can be selected from the group consisting of: sodium bicarbonate, sodium carbonate, sodium hydroxide, potassium carbonate, potassium bicarbonate, potassium hydroxide, ammonia or combinations thereof. In one embodiment, the acetate salt is contacted with ammonia (preferably aqueous ammonia, i.e., ammonium hydroxide) to produce the corresponding freebase.

Erdafitinib acetate can be manufactured for example using anti-solvent crystallization.

The present disclosure further provides a process for preparation of Erdafitinib or salt thereof, preferably substantially pure Erdafitinib or pure salt thereof, wherein the process comprises:
a) contacting Erdafitinib with formic acid to produce formate salt of Erdafitinib; and
b) converting the formate salt of Erdafitinib to Erdafitinib or another salt thereof.

In embodiments, Erdafitinib formate formed in step a) is crystalline, preferably Erdafitinib formate form B or D or a combination thereof.

Formation of the formate salt advantageously aids in removal of impurities prior to isolation of the freebase. The formate salt is subsequently converted back to the freebase form (step (i)) to produce Erdafitinib.

In particular, in step (b) the formate salt may be contacted with a base, such as an organic or inorganic base, to produce the corresponding freebase. The base may be any suitable alkaline, preferably selected from the group consisting of: sodium bicarbonate, sodium carbonate, sodium hydroxide, potassium carbonate, potassium bicarbonate, potassium hydroxide, cesium bicarbonate, cesium carbonate, cesium hydroxide, lithium bicarbonate, lithium carbonate, lithium hydroxide, ammonia, an organic amine, preferably a mono(C₁-C₆)alkylamine, a di(C₁-C₆)alkylamine, a tri(C₁-C₆)alkylamine), a tertiary(C₁-C₆)alkylamine, a C₅-C₁₀ heterocyclic amine, a C₅-C₁₀ arylamine, a C₄-C₈ heteroaryl amine, or combinations thereof. More particularly, the base can be selected from the group consisting of: sodium bicarbonate, sodium carbonate, sodium hydroxide, potassium carbonate, potassium bicarbonate, potassium hydroxide, ammonia or combinations thereof. In one embodiment, the formate salt is contacted with ammonia (preferably aqueous ammonia, i.e., ammonium hydroxide) to produce the corresponding freebase.

Erdafitinib formate can be manufactured for example using anti-solvent crystallization.

The present disclosure further provides a process for preparation of Erdafitinib or salt thereof, preferably substantially pure Erdafitinib or pure salt thereof, wherein the process comprises:
a) contacting Erdafitinib with methanesulfonic acid to produce mesylate salt of Erdafitinib; and
b) converting the mesylate salt of Erdafitinib to Erdafitinib or another salt thereof

In embodiments, Erdafitinib mesylate formed in step a) is crystalline, preferably Erdafitinib mesylate form F.

Formation of the mesylate salt advantageously aids in removal of impurities prior to isolation of the freebase. The mesylate salt is subsequently converted back to the freebase form (step (i)) to produce Erdafitinib. In particular, in step (b) the mesylate salt may be contacted with a base, such as an organic or inorganic base, to produce the corresponding freebase. The base may be any suitable alkaline, preferably selected from the group consisting of: sodium bicarbonate, sodium carbonate, sodium hydroxide, potassium carbonate, potassium bicarbonate, potassium hydroxide, cesium bicarbonate, cesium carbonate, cesium hydroxide, lithium bicarbonate, lithium carbonate, lithium hydroxide, ammonia, an organic amine, preferably a mono(C₁-C₆)alkylamine, a di(C₁-C₆)alkylamine, a tri(C₁-C₆)alkylamine), a tertiary(C₁-C₆) alkylamine, a C₅-C₁₀ heterocyclic amine, a C₅-C₁₀ arylamine, a C₄-C₈ heteroaryl amine, or combinations thereof. More particularly, the base can be selected from the group consisting of: sodium bicarbonate, sodium carbonate, sodium hydroxide, potassium carbonate, potassium bicarbonate, potassium hydroxide, ammonia or combinations thereof. In one embodiment, the mesylate salt is contacted with ammonia (preferably aqueous ammonia, i.e., ammonium hydroxide) to produce the corresponding freebase. Erdafitinib mesylate can be manufactured for example using anti-solvent crystallization.

The above-described processes for preparing or purifying Erdafitinib, are represented Scheme 1 below:

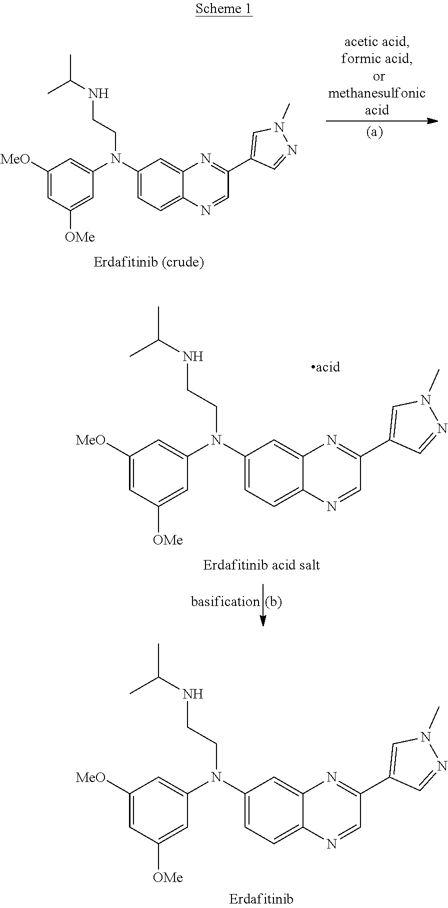 [SMILES: COc1cc(OC)cc(N(CCNC(C)C)c2ccc3ncc(-c4cnn(C)c4)nc3c2)c1 ; COc1cc(OC)cc(N(CCNC(C)C)c2ccc3ncc(-c4cnn(C)c4)nc3c2)c1 ; COc1cc(OC)cc(N(CCNC(C)C)c2ccc3ncc(-c4cnn(C)c4)nc3c2)c1]

wherein: a) Erdafitinib is contacted with acetic acid, formic acid or methane sulfonic acid to produce the acid addition salt Erdafitinib; and b) the acid addition salt of Erdafitinib is converted to Erdafitinib or another salt thereof, preferably to Erdafitinib, step (b) is carried out in a solvent selected from the group consisting of water, or a C₃ to C₈ ester or a combination thereof; preferably water or a C₃ to C₆ ester or a combination thereof, more preferably water, ethyl acetate, propylacetate, butyl acetate, isopropyl acetate, or isobutyl acetate or a combination of water and one of these esters; and most preferably water or isopropyl acetate, or a combination of water and isopropylacetate. Preferably, the solvent is a mixture of water and a C₃ to C₈ ester, preferably water and a C₃ to C₆ ester, or water and one of ethyl acetate, propylacetate, butyl acetate, isopropyl acetate, and more preferably water and isopropyl acetate. The water may be used in an amount of: about 5 ml to about 30 ml, about 8 ml to about 20 ml, about 10 ml to about 15 ml, or about 12 to about 13 ml, per gram of Erdafitinib salt. The ester is used in an amount of: about 5 ml to about 30 ml, about 8 ml to about 20 ml, about 10 ml to about 15 ml, or about 12 to about 13 ml, per gram of Erdafitinib salt. Particularly, the water and ester may be used in a ratio of (v/v): about 3:1 to about 1:3, about 2:1 to about 1:2, about 1.5:1 to about 1:1.5, about 1.2:1 to about 1:1.2, or about 1:1. The reaction may be carried out at a temperature of: about 20° C. to about 60° C., about 25° C. to about 40° C., about 30° C. to about 35° C. The base in step (b) may be used in an amount to provide a pH of: >8; preferably wherein the base is added in an amount of: about 1 to about 10, about 1 to about 8, about 1 to about 5, about 1.5 to about 3, or about 2 to about 2.6, or about 2.3 mole equivalents, relative to Erdafitinib. The Erdafitinib may be isolated by a process comprising solvent extraction using an organic solvent comprising a C₃ to C₈ ester, preferably a C₃ to C₆ ester, preferably ethyl acetate, propylacetate, butyl acetate, or isopropyl acetate; and more preferably isopropyl acetate, to provide a solution of Erdafitinib in the organic solvent. The solution may be filtered, optionally washed with water, concentrated, optionally cooled and optionally seeded, to precipitate Erdafitinib. The Erdafitinib may be isolated by any suitable procedure, for example: by decantation, centrifuge or filtration, preferably filtration. The Erdafitinib may be dried, optionally at a temperature of about 20° C. to about 60° C., about 25° C. to about 55° C., about 30° C. to about 50° C., or about 40° C. to about 45° C., and preferably under reduced pressure.

The Erdafitinib prepared by the process of any embodiment or aspect as described herein may be advantageously isolated in a purity of: at least about 99.8%, at least about 99.9%, at least about 99.95%, at least about 99.98%, at least about 99.99% or at about 100%, preferably as measured by HPLC According to any of the above-described processes for preparing or purifying Erdafitinib.

Particularly, the acid addition salt of Erdafitinib is Erdafitinib formate or Erdafitinib mesylate, preferably Erdafitinib formate. Accordingly, the present disclosure provides a process for the preparation of Erdafitinib or salt thereof, preferably substantially pure Erdafitinib or a substantially pure Erdafitinib salt, wherein the process comprises:
a) contacting Erdafitinib with formic acid to produce Erdafitinib formate; and
b) converting the formate salt of Erdafitinib to Erdafitinib or another salt thereof, preferably to Erdafitinib. Preferably, the Erdafitinib formate is crystalline; and optionally wherein the Erdafitinib formate is form B or form D, as described herein or a combination thereof.

Alternatively, any one of the Erdafitinib salts and their solid state forms disclosed herein can be converted to other salts by salt switching, i.e., reacting Erdafitinib acid addition salt, with an acid having a pKa which is lower than the pKa of the acid of the first Erdafitinib acid addition salt.

The present disclosure further encompasses processes for preparing Erdafitinib and intermediates. A general scheme (Scheme 2) showing a process for preparing compound 9, which is an intermediate of Erdafitinib is set out below:

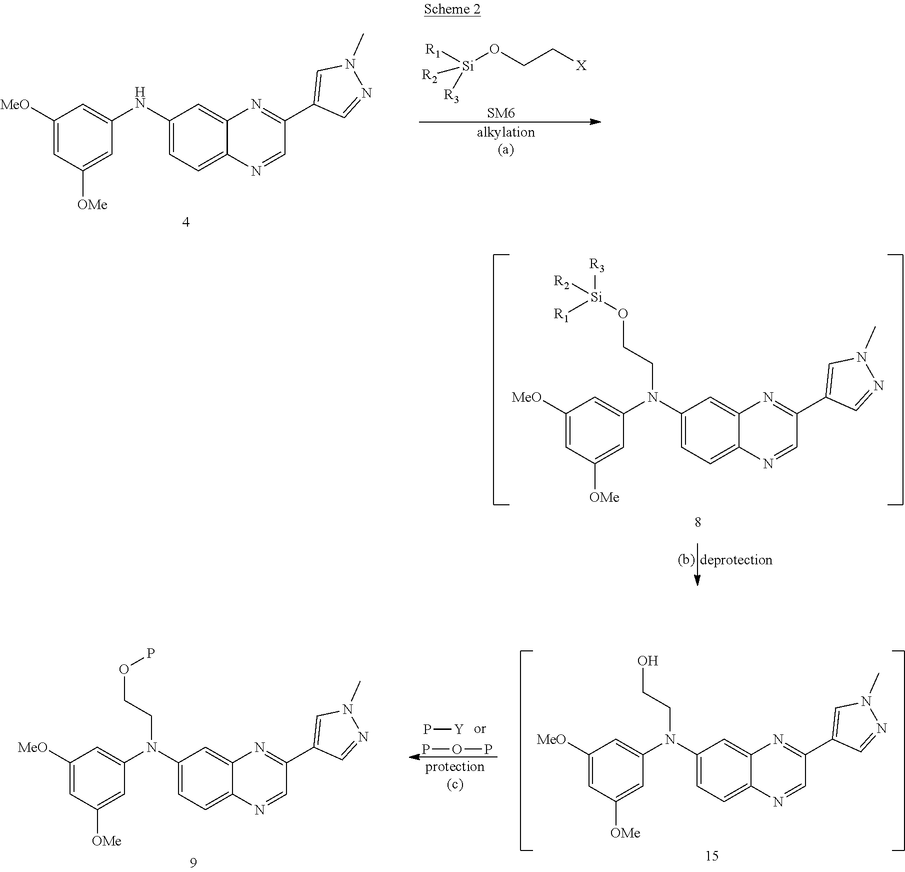

As can be seen in the above Scheme 2, compound 9 can be prepared in three reaction steps from compound 4, by: (a) alkylation of compound 4 with a silyl-protected halo ethanol compound SM6 wherein each R₁, R₂ and R₃ can be the same or different and each independently represents a C₁-C₆ alkyl, C₄-C₆ cycloalkyl or C₆-C₁₀ aryl; and X is halo, to form compound 8; (b) deprotection (i.e. desilylation) of compound 8 to form the compound 15, and (c) protecting the hydroxyl group in compound 15 by reaction with a compound P—Y or P—O—P, wherein P is a substituent selected from the group consisting of methanesulfonyl, ethanesulfonyl, benzenesulfonyl, p-toluenesulfonyl, p-bromobenzenesulfonyl, fluoromethanesulfonyl, or p-nitrophenylsulfonate; and Y is halo. Advantageously, the present process avoids the need to isolate the intermediate compounds 8 and 15 (denoted in Scheme 2 by the square parentheses), for example one or both (preferably both) of the intermediate compounds 8 and 15 may be obtained as solutions from the reaction work up procedures, with no need to purify by crystallisation or chromatography to form solid products.

the reaction in step (a) comprises:
(i) reacting the compound (4) with a base, preferably selected from the group consisting of: an alkali metal alkoxide, an alkali metal hydroxide or a alkali metal hydride to form a first mixture, and
(ii) combining the first mixture with the compound SM6.

The base in step (i) is preferably an alkali metal alkoxide; preferably sodium methoxide, sodium ethoxide, sodium tert-butoxide, potassium methoxide, potassium ethoxide, potassium tert-butoxide; more preferably sodium methoxide, sodium ethoxide, sodium tert-butoxide, and most preferably sodium tert-butoxide. The base in step (i) may be added in an amount of about: 1 to about 4, about 1.5 to about 3, about 1.8 to about 2.5, about 2 to about 2.1, or about 2.05, mole equivalents, relative to Compound 4. Step (i) may be carried out in a solvent selected from the group consisting of: acetonitrile, tetrahydrofuran, methyl-tetrahydrofuran, and preferably acetonitrile. Suitably, step (i) may be carried out at temperature of: about −10° C. to about 20° C., about −5° C. to about 10° C., about −2° C. to about 5° C., or about 0° C. Step (i) may be carried out in an inert atmosphere, preferably under nitrogen, or under argon, most preferably nitrogen. Step (i) may be carried out over a period of: about 0.2 hour to about 3 hours, about 0.5 hour to about 2 hours, or about 1 hour.

Preferably according to any aspect or embodiment of the processes disclosed herein, each R₁, R₂ and R₃ in compound SM6 can be the same or different and each independently represents a C₁-C₆ alkyl, and X is halo; more preferably each R₁, R₂ and R₃ can be the same or different and each independently represents a C₁-C₄ alkyl; and X is chloro or bromo; and most preferably the compound 6 is 2-bromoethoxy-tert-butyldimethylsilane.

Step (ii) particularly comprises adding compound SM6 to the reaction mixture in step (i). Step (ii) may be carried out at a temperature of: about 20° C. to about 60° C., about 30° C. to about 50° C., about 35° C. to about 45° C., or about 40° C. Step (ii) may be carried out over a period of: about 0.5 hour to about 5 hours, about 1 hour to about 4 hours, or about 1.5 to about 3 hours, or about 2.5 hours.

The compound 8 from the alkylation reaction of Scheme 2, step (a) is not isolated from the reaction mixture. That is, after work up, the compound 8 is maintained as a solution. The solution may be used directly in step (b) of Scheme 2. Step (b) comprises reacting the compound 8 from step (a) with a deprotecting agent, preferably a fluoride, selected from the group consisting of: tetra-n-butylammonium fluoride, or alkali metal fluorides or alkali metal hydrogen fluorides, preferably tetra-n-butylammonium fluoride, potassium fluoride, potassium hydrogen fluoride, and most preferably tetra-n-butylammonium fluoride (TBAF). The deprotecting agent can be added to the reaction mixture after step (i). The deprotecting agent is used in an amount of: about 1 to about 4, about 1.5 to about 3, about 2 to about 2.8, or about 2.5, mole equivalents, relative to compound 4. Preferably, step (b) is carried out at a temperature of: about 20° C. to about 60° C., about 30° C. to about 50° C., about 35° C. to about 45° C., or about 40° C. Any suitable reaction time can be used. For example, step (b) may be carried out over a period of: about 0.5 hour to about 24 hours, about 1 hour to about 8 hours, or about 1.5 to about 3 hours, or about 2.5 hours.

After the reaction in step (b), the reaction mixture is cooled, preferably to room temperature, and washed with an aqueous solution of an inorganic base, preferably selected from the group consisting of an alkali metal carbonate or an alkali metal hydrogen carbonate, preferably sodium carbonate, potassium carbonate, sodium hydrogen carbonate or potassium hydrogen carbonate, more preferably sodium carbonate or potassium carbonate, and most preferably potassium carbonate. The resulting compound 15 can be obtained by extraction of the reaction mixture in step (b), preferably using an extraction solvent selected from the group consisting of: toluene, m-xylene, acetonitrile, cyclohexane, and ethylacetate, and preferably toluene, to form a solution of compound 15 in the extraction solvent. Preferably, the solution of compound 15 from the extraction is azeotropically distilled to remove water to form a solution of compound 15 in the extraction solvent, wherein the water content is: 2% or less, 1.5% or less, 1% or less, or 0.5% or less. Advantageously in the process of Scheme 2, compound 15 from the deprotection reaction is obtained from the work up without further purification, and is maintained in solution.

Step (c) in Scheme 2 comprises combining the compound 15 from step (b) with the compound P—Y or P—O—P, at a temperature of 50° C. or less, about −10° C. to about 30° C., about 10° C. to about 28° C., about 15° C. to about 25° C., or about 20° C. to about 25° C. Optionally, prior to combining with the compound P—Y or P—O—P, the compound from step (b) is combined with an organic base, preferably an tertiary aliphatic or aromatic amine, or heterocyclic tertiary amine, particularly a tri(C₁₋₆)alkylamine, pyridine, more particularly trimethylamine, triethylamine, and most preferably triethylamine. The organic amine may be added in an amount of: about 1 to about 12, about 2 to about 10, about 4 to about 8, about 5 to about 7, or about 6 to about 6.5 mole equivalents relative to compound 4.

Preferably according to any aspect or embodiment of the processes disclosed herein, step (c) in Scheme 2 comprises reacting compound 15 with the compound P—Y. More preferably, the substituent P in P-Y is selected from the group consisting of methanesulfonyl, ethanesulfonyl, benzenesulfonyl, p-toluenesulfonyl; and Y is chloro or bromo. Most preferably, the compound P-Y is methanesulfonyl chloride.

Particularly, in step (c) of Scheme 2, the compound P—Y or P—O—P can be added to the compound 15 from step (b), preferably in a solution with an organic solvent. A suitable organic solvent may be selected from the group consisting of: acetone, toluene, acetonitrile, and preferably acetone.

Following the reaction of compound 15 with the compound P—Y or P—O—P, the pH of the reaction mixture is adjusted to: ≥7, preferably with an organic base, preferably a tertiary alkylamine, tertiary heterocyclic amine, or a tri(C₁₋₆)alkylamine, pyridine, particularly a trialkylamine, more particularly trimethylamine, triethylamine, and most preferably triethylamine. The reaction mixture may be cooled, preferably to a temperature of: about −10° C. to about 20° C., about 5° C. to about 15° C., about 8° C. to about 12° C., or about 10° C., and combined with water. Water may be added to the reaction mixture in a ratio (v/v) of water to reaction mixture of: about 5:1 to about 0.8: 1, about 2:1 to about 1:1, about 1.8:1 to about 1.3:1 or about 1.5:1. The reaction mixture may be stirred at a temperature of: about -10° C. to about 30° C., about 5° C. to about 22° C., about 8° C. to about 20° C., or about 10° C. to about 15° C.

The resulting compound 9 from step (c) may be isolated by any suitable method, preferably by filtration.

Advantageously, intermediate compound 8 and compound 15 in high purity may be obtained without crystallization or column chromatography. Accordingly, compound 8 and compound 15, can be maintained in solution and used in the subsequent steps as a solution. Compound 9, optionally as prepared according any of the processes described herein, may be converted to Erdafitinib by amination, preferably using isopropylamine or N-isopropylpropan-2-imine, and more preferably using isopropylamine.

Advantageously, the present process provides a process for preparing highly pure Erdafitinib starting from Compound 9, which is generally represented in Scheme 3:

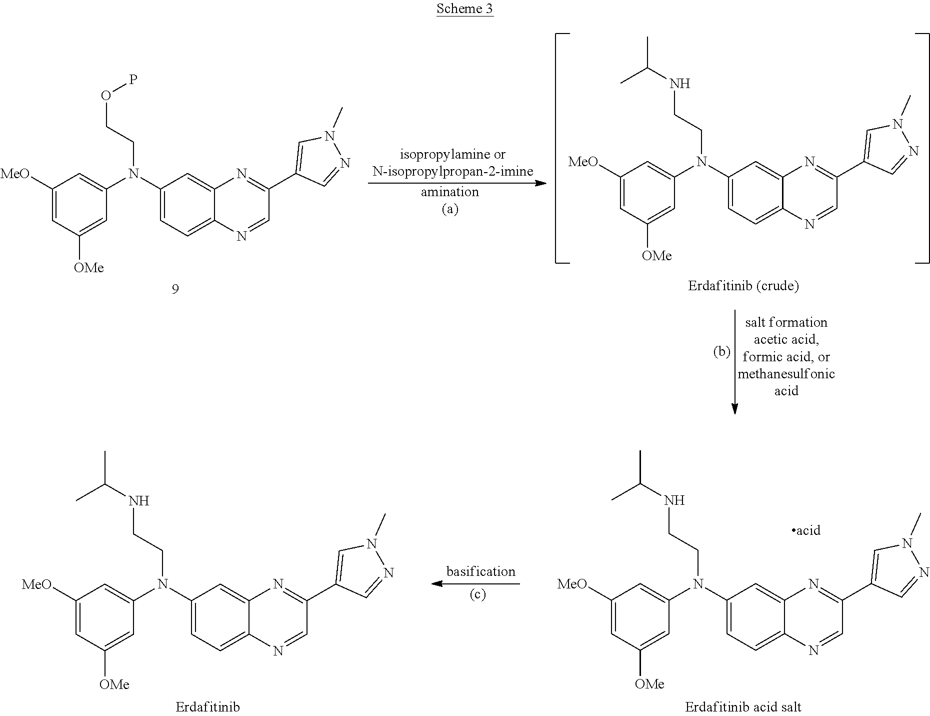

As illustrated in the above Scheme 3, highly pure Erdafitinib may be prepared by a process comprising (1) amination of compound 9 (wherein P is as defined above) with isopropylamine or N-isopropylpropan-2-imine. Typically, this reaction produces a crude product Erdafitinib. Advantageously, it has been found that purification of the crude Erdafitinib by salt formation with acetic acid, formic acid or methanesulfonic acid (preferably formic acid or methanesulfonic acid, followed by basification, can be conducted in high yield, to produce highly pure Erdafitinib, due to the surprising ability of the acetate, formate or methane sulfonate salts to purge the impurities typically present in the crude Erdafitinib intermediate.

Advantageously, the process of Scheme 3 can be conducted without isolation of the intermediate crude Erdafitinib (denoted in Scheme 3 by the square parentheses). For example, crude intermediate Erdafitinib may be obtained as a solution from the reaction work up procedure, with no need to purify by crystallisation or chromatography to form a solid or crystalline product.

In Scheme 3, step (a) may be carried out in a solvent selected from the group consisting of: water, acetonitrile, tetrahydrofuran, methyltetrahydrofuran, or combinations thereof, optionally wherein step (a) is carried out in a solvent selected from: acetonitrile and water. The solvent in step (a) may be a mixture of water and acetonitrile, optionally wherein the ratio (v/v) of water to acetonitrile is: about 1:8 to about 1:40, about 1:10 to about 1:30, about 1:15 to about 1:25, or about 1:20. The isopropylamine or N-isopropylpropan-2-imine can be used in an amount of: about 3 to about 30 moles, about 5 to about 25 moles about 10 to about 20 moles, about 12 to about 18 moles, or about 15 moles, per mole of compound 9.

Step (a) of Scheme 3 may be carried out at a temperature of: about 40° C. to about 120° C., about 60° C. to about 110° C., about 80° C. to about 100° C. or about 85° C. to about 90° C. Step (a) may be carried out under pressure, particularly under a pressure of: about 120 kPa to about 400 kPa, about 130 kPa to about 300 kPa, about 140 kPa to about 200 kPa, about 150 kPa to about 190 kPa, about 160 kPa to about 180 kPa, or about 170 kPa. Step (a) may be carried out over a period of: about 1 hour to about 16 hours, about 3 hours to about 12 hours, about 5 hours to about 10 hours, about 6 hours to about 8 hours, or about 7 hours.

After the amination reaction, the reaction mixture in step (a) of Scheme 3 can be concentrated by evaporation to form a first mixture comprising a concentrated solution comprising compound 9, and, if necessary, replacing the solvent in the concentrated solution with a solvent selected from the group consisting of: methyltetrahydrofuran, tetrahydrofuran, toluene, acetone, isopropylacetate, to form a second mixture comprising a solution of Erdafitinib crude in a solvent selected from the group consisting of: methyltetrahydrofuran, tetrahydrofuran, toluene, acetone, or isopropylacetate. The second mixture comprising a solution of Erdafitinib crude is washed with an aqueous solution of an inorganic base, preferably selected from the group consisting of an alkali metal carbonate or an alkali metal hydrogen carbonate, preferably sodium carbonate, potassium carbonate, sodium hydrogen carbonate or potassium hydrogen carbonate, more preferably sodium carbonate or potassium carbonate, and most preferably potassium carbonate. The second mixture or the second mixture following washing with an inorganic base, can be further washed one or more times with saturated brine. Optionally the mixture can be filtered after washing steps to remove any insoluble matter. The second mixture can be diluted with a solvent selected from the group consisting of: methyltetrahydrofuran, tetrahydrofuran or toluene, prior to filtering, preferably wherein the solvent is added in an amount of: about 10 ml to about 70 ml, about 20 ml to about 60 ml, about 30 ml to about 50 ml, about 35 to about 45 ml, or about 40 ml, per gram of Erdafitinib. Preferably the process is carried out without isolation of Erdafitinib and hence the Erdafitinib product after the amination reaction of step (a) is maintained in solution throughout.

In Scheme 3, step (b) comprises reacting the solution of Erdafitinib from step (a) with the acid, wherein the reaction is conducted at a temperature of: about 40° C. to about 100° C., about 50° C. to about 90° C., about 60° C. to about 80° C., about 65° C. to about 75° C. or about 70° C. The mixture in step (b) may be cooled to a temperature of: about −10° C. to about 20° C., about −5° C. to about 10° C., about −2° C. to about 5° C., or about 0° C. The reaction may be maintained for a period of about: 0.5 hour to about 10 hours, about 1 hour to about 8 hours, about 2 hours to about 5 hours, about 3 hours to about 4 hours, or about 3.5 hours. Following the reaction, the salt of Erdafitinib can be crystallized from the reaction mixture in step (b). The salt may be isolated by filtration of the reaction mixture in step (b).

The present disclosure provides a process for preparing Erdafitinib by carrying out the processes according to Scheme 2 and Scheme 3. Accordingly, Scheme 4 represents a process for preparing Erdafitinib starting from compound 4, wherein the steps (i), (ii), (iii), (iv) and (v) are carried out according to any embodiment of the alkylation, deprotection, protection, amination, salt formation, and basification steps as described herein:

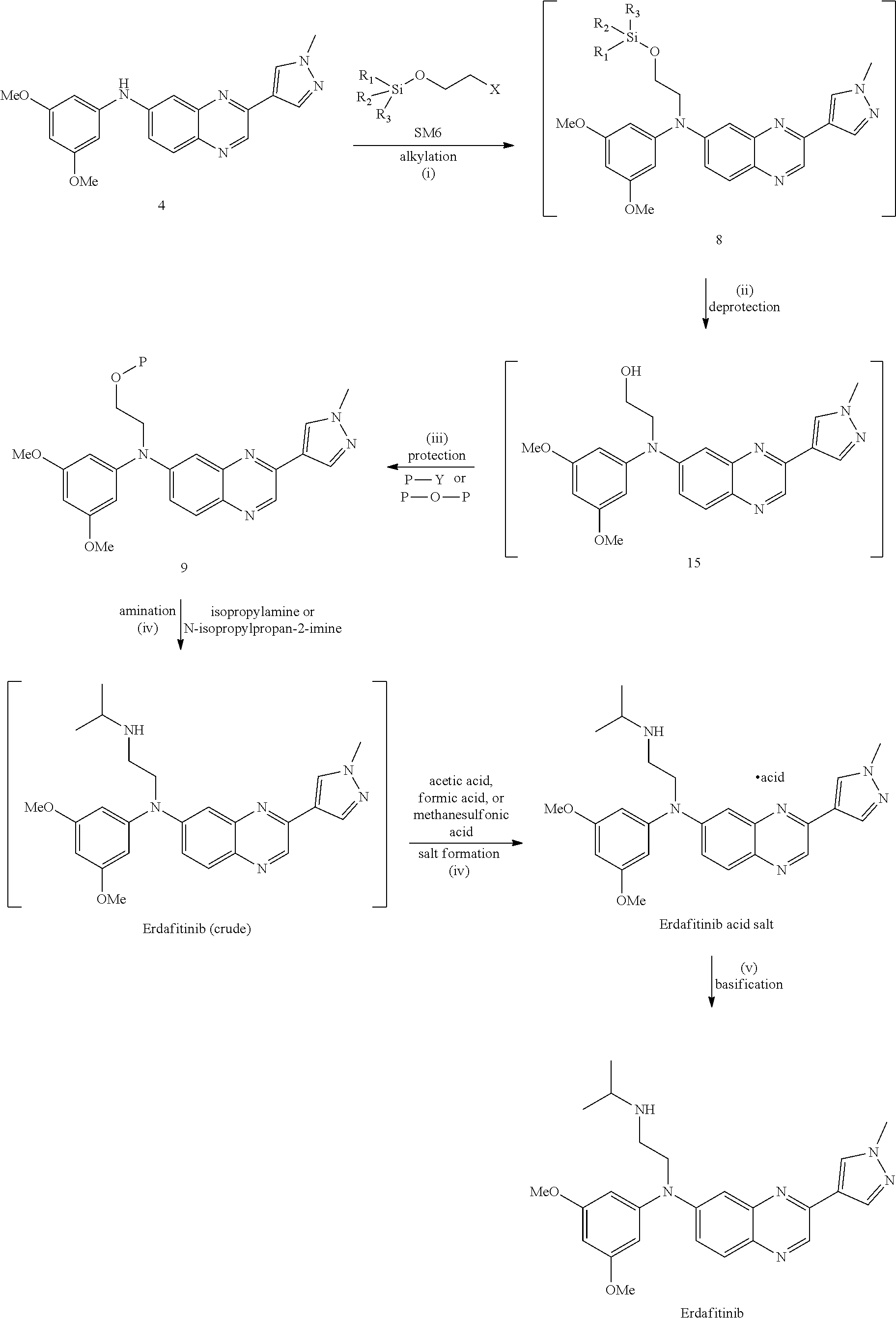

In a preferred process, the preparation of Erdafitinib can be carried out starting from compound 4, following Scheme 4, wherein intermediates, 8, 15 and crude Erdafitinib are not isolated. Advantageously the above process involves the isolation of only intermediates 9 and Erdafitinib acid salt as solids.

Advantageously the processes of the present disclosure enable the production of highly pure Erdafitinib or salts thereof (for example: having a purity of: at least 99.6%, at least 99.8%, at least 99.9%, at least 99.92%, at least 99.94%, at least 99.96%, at least 99.98% or about 100%). Surprisingly, the processes of the present disclosure are able to achieve such high purities without the need for column chromatography procedures or crystallization steps, which are undesirable on large scale and/or result in a lower yield.

The present disclosure encompasses processes to prepare said pharmaceutical formulations of Erdafitinib or Erdafitinib salts including combining the described solid state forms of Erdafitinib salts and at least one pharmaceutically acceptable excipient. Excipients are added to the formulation for a variety of purposes. Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present disclosure, Erdafitinib or Erdafitinib salts and any other solid excipients are dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present disclosure include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present disclosure can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, the most preferred route of the present disclosure is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, in embodiments a powdered or granulated solid composition of the disclosure, within either a hard or soft shell. The shell can be made from gelatin and optionally contain a plasticizer such as glycerin and sorbitol, and an opacifying agent or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can comprise any of the aforementioned blends and granulates that were described with reference to tableting, but they are not subjected to a final tableting step.

A pharmaceutical formulation of Erdafitinib or Erdafitinib salts disclosed herein can be administered. Erdafitinib or the Erdafitinib salts disclosed herein is preferably formulated for administration to a mammal, preferably a human, by oral administration. Erdafitinib or the Erdafitinib salts disclosed herein can be formulated, for example, as a tablet or capsule. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The solid state forms defined herein as well as the pharmaceutical compositions or formulations of the improved solid state form of Erdafitinib or the Erdafitinib salts disclosed herein can be used as medicaments, in embodiments for the treatment of cancer, as specified above.

The present disclosure also provides methods of treating medicaments, in embodiments for the treatment of cancer as specified above, by administering a therapeutically effective amount of the improved solid state form of Erdafitinib the Erdafitinib salts disclosed herein of the present disclosure, or at least one of the herein described pharmaceutical compositions or formulations, to a subject suffering from the above specified diseases, or otherwise in need of the treatment.

The present disclosure also provides uses of the improved solid state form of Erdafitinib the Erdafitinib salts disclosed herein of the present disclosure, or at least one of the above pharmaceutical compositions or formulations for the manufacture of medicaments, in embodiments for medicaments, in embodiments for the treatment of cancer, as specified above.

Having described the disclosure with reference to certain preferred embodiments, other embodiments will become apparent to one skilled in the art from consideration of the specification. The disclosure is further illustrated by reference to the following examples describing in detail the preparation of the composition and methods of use of the disclosure. It will be apparent to those skilled in the art that many modifications, both to materials and methods, may be practiced without departing from the scope of the disclosure.

### Analytical Methods

### XRPD Method:

Powder X-ray Diffraction was performed on an X-Ray powder diffractometer PanAlytical X′pert Pro; CuKα radiation (λ=1.54187 Å); X′Celerator detector with active length 2.122 degrees 2-theta; laboratory temperature 25±3° C.; zero background sample holders. Prior to analysis, the samples were gently ground using a mortar and pestle to obtain a fine powder. The ground sample was adjusted into a cavity of the sample holder and the surface of the sample was smoothed using a cover glass.

### Measurement Parameters:

| Scan range | 3-40 degrees 2-theta | |
|---|---|---|
| Scan mode | continuous | |
| Step size | 0.0167 | degrees |
| Step size | 42 | s |
| Sample spin | 60 | rpm |

| | |
|---|---|
| Sample holder | zero background silicon plate |

### HPLC Method Used for Purity Analysis of Example 1, Procedure A

| | | | |
|---|---|---|---|
| Column: | Kinetex EVO C18 100 Å | Particle size [μm]: | 1.7 |
| Column dim. [mm]: | 100 × 2.1 | Flow-rate [ml/min]: | 0.5 |
| Column temp. [° C.]: | 45 | Sample temp. [° C.]: | 20 |
| Detection UV [nm]: | 220, 238 | Injection volume [μl]: | 1.0 |

| Mobile phase | Solvent A: | | | |
|---|---|---|---|---|
| | Prepare a premixed solvent of ammonium formate - MeCN 95:5 (v/v). Add 400 | | | |
| | μL of formic acid to 950 mL of degassed water (10.8 mmolL⁻¹), adjust pH with | | | |
| | ammonia to 4.6 and add 50 mL of MeCN. Mix solution well. | | | |
| | Solvent B: | | | |
| | Prepare a premixed solvent of ammonium formate - MeCN 10:90 (v/v). Add 100 | | | |
| | mL of 27 mmolL⁻¹ of ammonium formate (500 μL of formic acid to 500 mL of | | | |
| | degassed water, adjust pH with ammonia to 4.6) to 900 mL of MeCN. Mix solution | | | |
| | well. | | | |
| | Gradient | Time/min | % A | % B |
| | | 0 | 87 | 13 |
| | | 0.5 | 87 | 13 |
| | | 12 | 5 | 95 |
| | | 13 | 5 | 95 |
| | | 13.1 | 87 | 13 |
| | | 16.0 | 87 | 13 |

| | |
|---|---|
| | common max pressure: ca 250 bar (Classic Acquity, UPLC12) |
| Diluent | MeCN |
| Blank | Diluent |
| ID solution | — |
| Sample solution | 1.5 mg/mL of Erdafitinib in diluent |
| Sensitivity test | inject 2000-times diluted sample solution (0.05% level) s/n ≤10 |
| Suitability test | — |
| (ID solution) | |

### HPLC Method Used for Purity Analysis of Erdafitinib for Examples 2 and 3

| | | | |
|---|---|---|---|
| Column: | XBridge Shield RP18 | Particle size [μm]: | 3.5 |
| Column dim. [mm]: | 150 × 3.0 | Flow-rate [ml/min]: | 0.7 |
| Column temp. [° C.]: | 40 | Sample temp. [° C.]: | 20 |
| Detection UV [nm]: | 230 | Injection volume [μl]: | 1.0 |

| Mobile phase | Solvent A: | | | |
|---|---|---|---|---|
| | 1 L H₂O + 0.15 ul/l HCOOH + ammonia to pH 5.5 | | | |
| | Solvent B: | | | |
| | MeCN | | | |
| | Gradient | Time/min | % A | % B |
| | | 0 | 95 | 5 |
| | | 1 | 95 | 5 |
| | | 13 | 5 | 95 |
| | | 15 | 5 | 95 |
| | | 15.1 | 95 | 5 |
| | | 18 | 95 | 5 |

| | |
|---|---|
| | common max pressure: ca 250 bar (Classic Acquity, UPLC12) |
| Diluent | MeCN |
| Blank | Diluent |
| Sample solution | 1.5 mg/mL of Edafitinib in diluent |
| Sensitivity test | inject 2000-times diluted sample solution (0.05% level) s/n ≥10 |

### HPLC Method Used for Purity Analysis of Example 4

| | | | |
|---|---|---|---|
| Column: | XBridge Shield RP18 (max. 400 bar) | Particle size [μm]: | 3.5 |
| Column dim. [mm]: | 150 × 3.0 | Flow-rate [ml/min]: | 0.7 |
| Column temp. [° C.]: | 40 | Sample temp. [° C.]: | 20 |
| Detection UV [nm]: | 257 | Injection volume [μl]: | 1 |

| Mobile phase | Solvent A: 1 L H₂O + 0.15 μL/L formic acid + ammonia to pH 5.5 | | | |
|---|---|---|---|---|
| | Solvent B: MeCN | | | |
| | Gradient | Time/min | % A | % B |
| | | 0 | 95 | 5 |
| | | 1 | 95 | 5 |
| | | 13 | 5 | 95 |
| | | 15 | 5 | 95 |
| | | 15.1 | 95 | 5 |
| | | 18 | 95 | 5 |

| | |
|---|---|
| | common max pressure: ca 260 bar (H-class, UPLC03) |
| Diluent | MeCN |
| Blank | Diluent |

### HPLC Method Used for Purity Analysis of Examples 5, 7-10

| | | | |
|---|---|---|---|
| Column: | Acquity UPLC BEH Shield RP18 | Particle size [μm]: | 1.7 |
| Column dim. [mm]: | 100 × 2.1 | Flow-rate [ml/min]: | 0.45 mL/min |
| Column temp. [° C.]: | 40 | Sample temp. [° C.]: | 20 |
| Detection UV [nm]: | 257 | Injection volume [μl] | 1.0 |

| Mobile phase | Solvent A: | | | |
|---|---|---|---|---|
| | 1 L H₂O + 300 ul/l HCOOH + ammonia to pH 4.6 | | | |
| | Solvent B: | | | |
| | MeCN | | | |
| | Gradient | Time/min | % A | % B |
| | | 0 | 80 | 20 |
| | | 0.5 | 80 | 20 |
| | | 25 | 50 | 50 |
| | | 29 | 15 | 85 |
| | | 30 | 15 | 85 |
| | | 30.1 | 80 | 20 |
| | | 35 | 80 | 20 |

| | |
|---|---|
| | common max pressure: ca. 600 bar (Waters Acquity H-class) |
| Diluent | MeCN |
| Blank | Diluent |
| Sample solution | 1.5 mg/mL of Erdafitinib in diluent |
| Sensitivity test | inject 2000-times diluted sample solution (0.05% level) s/n ≥10 |

### HPLC Method Used for Purity Analysis in Example 6

| | | | |
|---|---|---|---|
| Column: | Acquity UPLC BEH Shield RP18 | Particle size [μm]: | 1.7 |
| Column dim. [mm]: | 100 × 2.1 | Flow-rate [ml/min]: | 0.45 mL/min |
| Column temp. [° C.]: | 40 | Sample temp. [° C.]: | 20 |
| Detection UV [nm]: | 257 | Injection volume [μl]: | 1.0 |

| Mobile phase | Solvent A: | | | |
|---|---|---|---|---|
| | 1 L H₂O + 300 ul/l HCOOH + ammonia to pH 4.6 | | | |
| | Solvent B: | | | |
| | MeCN | | | |
| | Gradient | Time/min | % A | % B |
| | | 0 | 80 | 20 |
| | | 0.5 | 80 | 20 |
| | | 25 | 50 | 50 |
| | | 29 | 15 | 85 |
| | | 30 | 15 | 85 |
| | | 30.1 | 80 | 20 |
| | | 35 | 80 | 20 |

| | |
|---|---|
| | common max pressure: ca. 600 bar (Waters Acquity H-class) |
| Diluent | MeCN |
| Blank | Diluent |
| Sample solution | 0.8 mg/mL of Erdafitinib in diluent |
| Sensitivity test | inject 2000-times diluted sample solution (0.05% level) s/n ≥10 |

### EXAMPLES

The starting material Erdafitinib can be prepared by any known method, for example as disclosed in Example B3 of International Publication No. WO 2011/135376, or as described in the examples below. The compound 4 starting material can be prepared by any known method, for example, by the methods disclosed in U.S. Pat. No. 8,895,601.

### Example 1

### Preparation of Form P of Erdafitinib Acetate

### Procedure A

1.9 grams of Erdafitinib (HPLC purity 88.7%) was dissolved in refluxing acetone (16 mL). Acetic acid (1 mol. eq., 0.249 ml) was added. Mixture was left spontaneously to cool to room temperature while stirred overnight. After 20 hours, thick suspension was filtered and product was washed with acetone (2 ml) yielding 1.296 g (59%) of yellow Erdafitinib acetate. The obtained solid was analyzed by XRPD and the XRPD pattern is presented in FIG. 1. HPLC purity 97.81%.

### Example 2

### Preparation of Form B of Erdafitinib Formate

Erdafitinib (HPLC purity 97.80%, 1.0 grams) was suspended in acetone (10 mL). The solid phase was dissolved by heating to 50° C. in 20 mL glass probe with magnetic stirring (500 rpm). Formic acid (103 mg-1 eq) was then added at 50° C. The solution was left to cool down until it reached a temperature of 45° C. and further cooled down according to linear ramp from 45° C. to (−5) ° C. over 60 minutes. The suspension was left to stand in refrigerator until the next day without stirring. In the morning the crystalline phase was blended, separated by filtration and washed with 5 mL of acetone. The cake was dried up on the filter at room temperature during 3 hours. Yield 0.92 grams (83%), HPLC purity 99.83%. The obtained solid was analyzed by XRPD and the XRPD pattern is presented in FIG. 2.

### Example 3

### Preparation of Form D of Erdafitinib Formate

Erdafitinib (HPLC purity 97.80%, 1.0 grams) was suspended in 2-methyl tetrahydrofurane (59 mL). The solid phase was dissolved by heating to 65° C. in 100 mL glass reactor with PBT impeller (300 rpm). Formic acid (103 mg-1 eq) was then added at 65° C. The solution was left to cool down until it reached a temperature of 58° C. At this point the solution was seeded (with about 4 mg of Form B Erdafitinib formic acid salt, prepared according to Example 2). Further cooled down according to linear cooling ramp from 58° C. to (−5° C.) over 150 minutes. The product gradually crystallized from the moment of seeding. The refrigerated suspension was left to stand in (at 0° C. in multireactor station) until the next day without stirring. In the morning the crystalline phase was blended, separated by filtration and washed with 10 mL of 2-Me-THF. The cake was dried up on the filter at room temperature during 3 hours. Yield 0.96 grams (88%), HPLC purity was 99.96%. The obtained solid was analyzed by XRPD and the XRPD pattern is presented in FIG. 3.

### Example 4

### Preparation of Form F of Erdafitinib Mesylate

500 mg of Erdafitinib (purity: 98.47% by HPLC) was suspended in 6.2 ml acetone at RT, during 30 min heated up to 50° C., clear solution was cooled down to 20° C. during 30 minutes. At 20° C. 7511.1 (1 eq.) methanesulfonic acid was added. After 15 minutes at 20° C., suspension was cooled down to 5° C. and stirred for 1 hour. The suspension was filtered and dried at RT under vacuum during 10 minutes. The obtained solid (purity: 99.15% by HPLC) was analyzed by XRPD and the XRPD pattern is presented in FIG. 4.

### Example 5

### Preparation of Erdafitinib Formate without Isolation of Erdafitinib

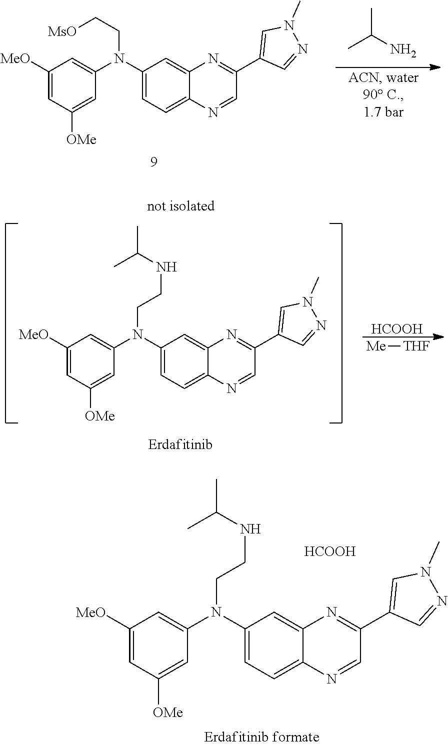

Compound 9 (purity: 99.5% by HPLC, 15 grams, 31.02 mmol) was suspended in a mixture of acetonitrile (150 ml) and water (7.5 ml) and isopropylamine (27.5 grams, 465.3 mmol) were added. The mixture was heated to 90° C. and stirred at 85-90° C. at a pressure of about 1.7 bars for 7 hours. The reaction mixture was swapped to Me-THF (150 ml) and extracted with 20% aqueous potassium carbonate and brine. The organic layer was diluted with Me-THF (480 ml) and clarified by filtration. (non-isolated Erdafitinib in organic layer of Me-THF, purity 97.63% by HPLC) The filtrate was heated to about 70° C. and formic acid 98% (2.16 g, 46.53 mmol) was added. The mixture was cooled to about 0° C. for about 3.5 hours. The crystalline solid was filtered, and the filter cake was rinsed with Me-THF. The product was dried to give 14.5 grams (95.1%) of Erdafitinib formate having a purity of 99.6% by HPLC.

### Example 6

### Preparation of Compound 9 from Compound 4 without Isolation of Intermediates

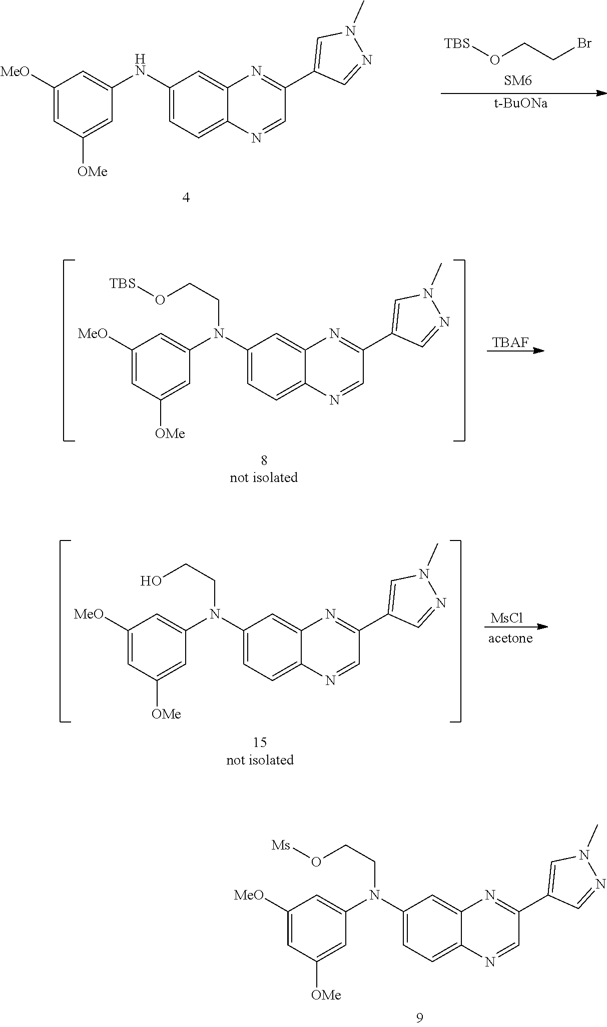

Compound 4 (700 grams, 1.94 mol), was diluted in acetonitrile (AcCN) (9100 ml, 13 ml/g) and cooled to 0° C. The reactor was properly inertized. Meanwhile, t-BuONa (382 grams, 2.05 eq) was suspended in AcCN (2100 ml, 3 ml/g) and added to the cooled reaction mixture. The reaction mixture was stirred at 0° C. for 1 hour. SM6 (781 grams, 1.50 eq) was added. The reaction mixture was heated to 40° C. over 2.5 hour linearly. Tetra-n-butylammonium fluoride TBAF.3H ₂O (1,528 grams, 2.50 eq) was added. After the reaction was complete, the reaction mixture was cooled at 20° C. The reaction mixture was extracted with 20 wt % aqueous solution of K₂CO₃ (7000 ml, 3 times) and toluene (10.5 L). The organic layer was distilled at 80° C. under reduced pressure to about half volume Additional toluene was added and the mixture concentrated several times under reduced pressure until the water content in the concentrate was less than 1%. The reaction mixture (concentrate) was diluted with acetone (13,300 ml, 19 ml/g) and triethylamine (1,225 ml, 6.25 eq) was added. A mixture of mesyl chloride (832 ml, 3.75 eq) in acetone (700 ml, 1 ml/g) was prepared and was slowly added to the reactor while cooling to not exceed 30° C. in the reaction mixture. The reaction mixture was stirred at 25° C. and the pH was adjusted to >8 as necessary using triethylamine. The reaction was checked for completion and if residual compound 15 was identified, triethylamine and mesyl chloride were added (1.5 mol equiv relative to residual compound 15). The mixture was cooled to 10° C. and water (21 L; 1.5×V of the reaction mixture) was slowly added, and the reaction mixture was stirred for the 1 hour at 10-15° C. The product was separated by filtration, and the reactor and filter cake were washed with the mixture of acetone:water (2:3) mixture (17.5 L) in three portions and cyclohexane (2×1.75 L). The product Compound 9 was dried was dried on filter using vacuum overnight, and then dried with a nitrogen stream at 50-55° C. until constant weight was reached purity 97.55% by HPLC).

### Purification of Compound 9:

Compound 9 (685 grams, 1.42 mol) was purified by slurrying in the mixture of toluene (5,137.5 ml, 7.5 ml/g) and acetone (1,712.5 ml, 2.5 ml/g). Triethylamine (8.22 ml, 0.012 ml/g) was added. The mixture was heated at 80° C. for 1 hour and then the suspension was cooled down to 0-5° C. over 30 minutes. The product was separated by filtration, and the filtration cake was washed with toluene (2×1370 ml,). The product was dried under vacuum at a temperature of up to 50° C. until constant weight, (purity 99.65%).

### Example 7

### Conversion of Compound 9 to Erdafitinib Crude

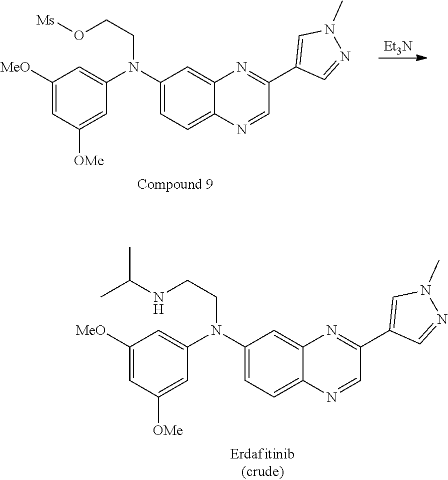 [SMILES: "Et3N" ; COc1cc(OC)cc(N(CCOS(C)(=O)=O)c2ccc3ncc(-c4cnn(C)c4)nc3c2)c1 ; COc1cc(OC)cc(N(CCNC(C)C)c2ccc3ncc(-c4cnn(C)c4)nc3c2)c1]

A mixture of compound 9 (270 grams, 0.56 mol) and isopropylamine (1680 mL, 19.5 mol) was stirred and heated at 80-90° C. in an autoclave reactor for 4 hours. The reaction mixture was cooled to room temperature and concentrated to about half volume. An aqueous ammonia 25% (63 mL, 0.84 mol) was slowly added and the mixture was cooled to about 15° C. The crystalline solid was filtered and the filter cake was rinsed with mixture of (tert-butyl methyl ether) TBME and acetonitrile (ACN) and then with water. The filter cake was dried under vacuum at about 50° C., to give 225 grams (90.3%) of Erdafitinib (crude), having a purity 99.5% by HPLC.

### Example 8

### Conversion of Compound 9 to Erdafitinib Crude

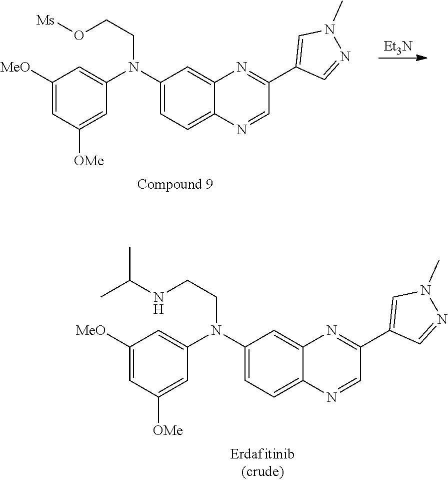 [SMILES: "Et3N" ; COc1cc(OC)cc(N(CCOS(C)(=O)=O)c2ccc3ncc(-c4cnn(C)c4)nc3c2)c1 ; COc1cc(OC)cc(N(CCNC(C)C)c2ccc3ncc(-c4cnn(C)c4)nc3c2)c1]

A mixture of Compound 9 (345 grams, 0.71 mol) and isopropylamine (2070 mL, 24.1 mol) was stirred and heated at 80-90° C. in an autoclave reactor for 4 hours. The reaction mixture was cooled to room temperature and concentrated to about half volume. An aqueous ammonia 25% (80 mL, 1.06 mol) was slowly added and the mixture was cooled to about 15° C. The crystalline solid was filtered and the filter cake was rinsed with mixture of AcCN and MTBE (1: 2; 2×225 ml) and then with water. The filter cake was dried under vacuum at about 50° C. to constant weight, to give 280 grams (87.9%) of Erdafitinib (crude, 99.09%) by HPLC.

### Example 9

### Conversion of Erdafitinib Crude to Erdafitinib Formate

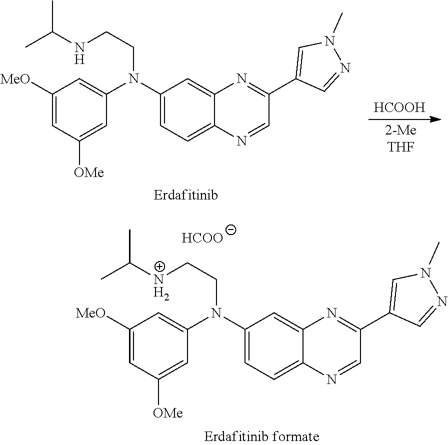

Erdafitinib crude (272 grams; 0.61 mol) was dissolved in methyl-tetrahydrofuran (Me-THF) (10.88 L, 40V). The solution was heated at 68° C. Formic acid (28 grams, 1.0 eq) was added as solid, and the mixture cooled to 61° C. The solution was seeded with Erdafitinib formate form D (0.68 grams), and the mixture was stirred at 61° C. for 30 minutes and then cooled down with linear ramp at −10° C./180 min in duplication. After the ending of the ramp, the mixture was cooled at −5° C. The mixture was stirred at this temperature for 1 hour. The product was filtered off, and the reactor and filter cake were washed with cool MeTHF (1360 ml; 5V). The product Erdafitinib formate was dried on filter under vacuum overnight, then discharged to vacuum drier and dried under vacuum and slight nitrogen stream at 35° C. until constant weight (yield 89%) purity 99.77% by HPLC. The obtained material was analyzed by XRPD and identified as Erdafitinib Formate form D.

### Example 10

### Conversion of Erdafitinib Formate to Erdafitinib Pure

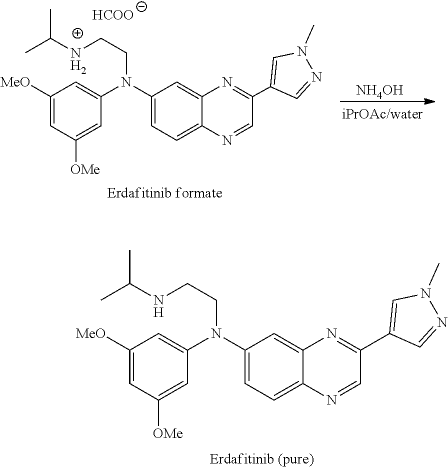

Erdafitinib formate (441 grams, 0.89 mol) was suspended in purified water (5.52 L, 12.5 ml/g). Isopropylacetate (5.52 L, 12.5 ml/g) was added and the system was properly inertized. The mixture was heated at 30-35° C. and stirred until all material was dissolved. An aqueous ammonia solution 25% (154 ml, 2.30 eq) was added (pH>8). The mixture was extracted with isopropylacetate/water and the organic layer was distilled under reduced pressure to about 3.9 L (8.7-9.0 V/SM) while heating at 60-70° C. The mixture was then heated at 75-80° C. under atmospheric pressure, and stirred until the solids were dissolved. The solution was cooled to −5° C. over about 3 hours. During cooling, when the mixture reached a temperature of 65° C., it was seeded with Erdafitinib crystals (3.3 grams, 0.005 g/g) (the seeding with Erdafitinib crystals is optional). The mixture was stirred at −5° C. for 1 hour, and the product was separated by filtration, washed with cooled isopropylacetate (670 ml; 1.5V) and dried. (yield 85%). Purity 99.95% by HPLC.

1. Erdafitinib formate.

2. The Erdafitinib formate according to claim 1, which is crystalline.

3. The Erdafitinib formate according to claim 2, designated Form B, which is characterized by data selected from one or more of the following: an XRPD pattern having peaks at 5.6, 8.7, 11.2, 14.5, and 15.4 degrees 2-theta±0.2 degrees 2-theta; or an XRPD pattern substantially as depicted in FIG. 2.

4. The Erdafitinib formate according to claim 3, which is characterized by: an XRPD pattern having peaks at 5.6, 8.7, 11.2, 14.5 and 15.4 degrees 2-theta±0.2 degrees 2-theta, and also having one, two, three, four or five additional peaks selected from 12.2, 18.2, 19.7, 22.0, and 24.6 degrees two theta±0.2 degrees two theta; or an XRPD pattern having peaks at 5.6, 8.7, 11.2, 12.2, 14.5, 15.4, 18.2, 19.7, 22.0, and 24.6 degrees 2-theta±0.2 degrees 2-theta.

5. The Erdafitinib formate according to claim 2, designated Form D, which is characterized by data selected from one or more of the following: an XRPD pattern having peaks at 6.9, 8.0, 13.4, 17.8, and 20.7 degrees 2-theta±0.2 degrees 2-theta; or an XRPD pattern substantially as depicted in FIG. 3.

6. The Erdafitinib formate according to claim 5, which is characterized by an XRPD pattern having peaks at 6.9, 8.0, 13.4, 17.8 and 20.7 degrees 2-theta±0.2 degrees 2-theta, and also having one, two, three, four or five additional peaks selected from 16.8, 21.4, 22.5, 23.8 and 24.6 degrees two theta±0.2 degrees two theta; or an XRPD pattern having peaks at 6.9, 8.0, 13.4, 16.8, 17.8, 20.7 21.4, 22.5, 23.8, and 24.6 degrees 2-theta±0.2 degrees 2-theta.

7-22. (canceled)

23. A process for the preparation of Erdafitinib or a salt thereof, wherein the process comprises:
a) contacting Erdafitinib with acetic acid, formic acid or methane sulfonic acid to produce the acid addition salt Erdafitinib; and
b) converting the acid addition salt of Erdafitinib to Erdafitinib or another salt thereof.

24-38. (canceled)

39. The process according to claim 23, for the preparation of Erdafitinib or salt thereof, wherein the process comprises:
a) contacting Erdafitinib with formic acid to produce Erdafitinib formate; and
b) converting the formate salt of Erdafitinib to Erdafitinib or another salt thereof.

40. The process according to claim 39, wherein the Erdafitinib formate is crystalline; and optionally wherein the Erdafitinib formate is form B or form D, or a combination thereof.

41. The process for preparing a formate salt of Erdafitinib comprising:
(a) amination of a compound of formula 9: 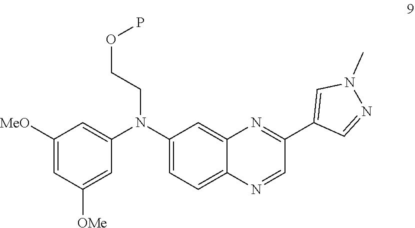
with isopropylamine or N-i sopropylpropan-2-imine;
wherein P is selected from the group consisting of methanesulfonyl, ethanesulfonyl, benzenesulfonyl, p-toluenesulfonyl, p-bromobenzenesulfonyl, fluoromethanesulfonyl, or p-nitrophenylsulfonate;
to form Erdafitinib: 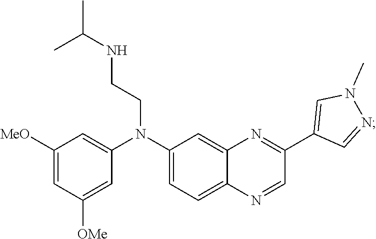 [SMILES: COc1cc(OC)cc(N(CCNC(C)C)c2ccc3ncc(-c4cnn(C)c4)nc3c2)c1]
and
(b) reacting Erdafitinib with formic acid;
wherein the process is carried out without isolation of Erdafitinib.

42-61. (canceled)

62. A process for preparing Compound 9: 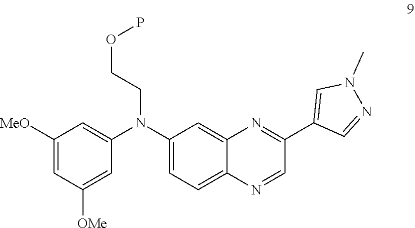
wherein P is a substituent selected from the group consisting of methanesulfonyl, ethanesulfonyl, benzenesulfonyl p-toluenesulfonyl, p-bromobenzenesulfonyl, fluoromethanesulfonyl, or p-nitrophenylsulfonate;
comprising:
(a) alkylation of Compound 4: 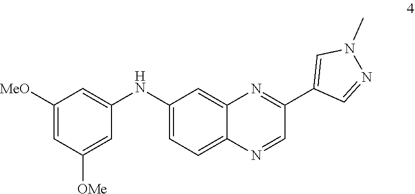 [SMILES: COc1cc(Nc2ccc3ncc(-c4cnn(C)c4)nc3c2)cc(OC)c1]
with a compound of formula SM6: 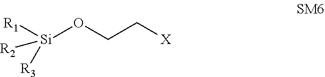
wherein each R₁, R₂ and R₃ can be the same or different and each independently represents a C₁-C₆ alkyl, C₄-C₆ cycloalkyl or C₆-C₁₀ aryl; and X is halo, to form Compound 8: 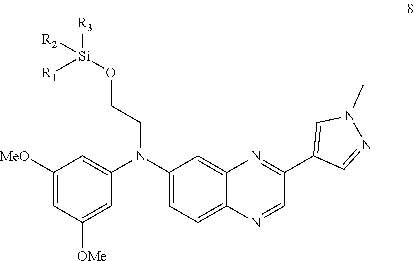
b) deprotecting compound 8 to form compound 15: 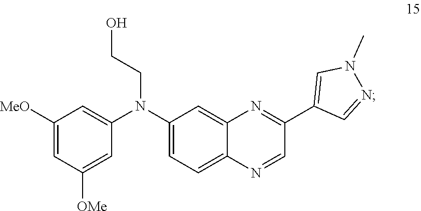 [SMILES: COc1cc(OC)cc(N(CCO)c2ccc3ncc(-c4cnn(C)c4)nc3c2)c1]
and
c) reacting the compound 15 with a compound P—Y, wherein P is as defined above, and Y is halogen, or a compound P—O—P, wherein P is as defined above;
wherein the process is carried out without isolation of compound 8 and/or compound 15.

63-94. (canceled)

95. The process according to claim 62, wherein the compound 9 is converted to a formate salt of Erdafitinib.

96. The process according to claim 95, further comprising converting the formate salt of Erdafitinib, to Erdafitinib by a process comprising contacting the salt of Erdafitinib with an organic or inorganic base, optionally wherein the base is selected from sodium bicarbonate, sodium carbonate, sodium hydroxide, potassium carbonate, potassium bicarbonate, potassium hydroxide, ammonia or combinations thereof.

97-109. (canceled)

110. The process according to claim 62, wherein P is selected from methanesulfonyl, ethanesulfonyl, benzenesulfonyl, or p-toluenesulfonyl.

111. The process according to claim 62, wherein R₁, R₂, R₃ are the same or different and each independently represents C₁-C₆ alkyl.

112. The process according to claim 62, wherein X is fluoro, chloro, bromo, or iodo.

113. The process according to claim 62, wherein Y is fluoro, chloro, bromo, or iodo.

114. The process according to claim 62, wherein Compound 9 has the formula:

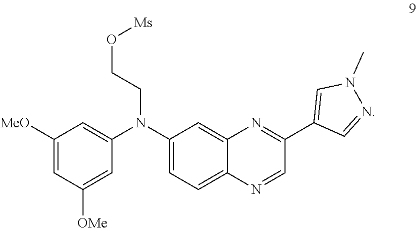 [SMILES: COc1cc(OC)cc(N(CCOS(C)(=O)=O)c2ccc3ncc(-c4cnn(C)c4)nc3c2)c1]

115. The process according to claim 62, wherein Compound 8 has the formula:

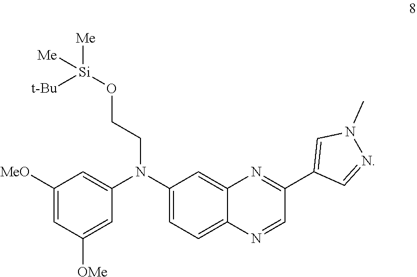 [SMILES: COc1cc(OC)cc(N(CCO[Si](C)(C)C(C)(C)C)c2ccc3ncc(-c4cnn(C)c4)nc3c2)c1]

116-118. (canceled)

119. The process according claim 23, further comprising combining the Erdafitinib with at least one pharmaceutically acceptable excipient to form a pharmaceutical composition.
